(19) United States
(12) Patent Application Publication
(10) Pub. No.: US 2024/0084012 A1
(43) Pub. Date: Mar. 14, 2024

(54) ANTI-PD-1/CD47 BISPECIFIC ANTIBODY AND USE THEREOF

(71) Applicant: ABVISION, INC., Reno, NV (US)

(72) Inventors: Chun-Jen LIN, San Jose, CA (US); Cheng-Chi CHAO, San Jose, CA (US); Chang-Hsin Chen, San Jose, CA (US); Gloria Guohong ZHANG, Reno, NV (US); Guochen YAN, Reno, NV (US)

(21) Appl. No.: 18/260,214

(22) PCT Filed: Dec. 31, 2021
(86) PCT No.: PCT/US21/65842

§ 371 Date: Jun. 30, 2023

Related U.S. Application Data
(60) Provisional application No. 63/133,126, filed on Dec. 31, 2020.

(51) Int. Cl. C07K 16/28; A61P 35/00

(52) U.S. Cl. CPC C07K 16/2818; A61P 35/00; C07K 16/2803; A61K 2039/505

(57) ABSTRACT

An isolated bispecific antibody or antigen-binding portion thereof includes a first chain which specifically binds to human PD-1(hPD-1) and blocks the interaction between hPD-1 and PD-L1, and a second chain which specifically binds to human CD47 and inhibits its interaction with SIRP-alpha, where the first chain and the second chain are coupled in a knob-in-hole format through their respective CH3 domain.

### BACKGROUND OF THE INVENTION

The protein Programmed Death 1 (PD-1) is an inhibitory member of the CD28 family of receptors, which also includes CD28, CTLA-4, ICOS and BTLA (Okazaki, et al. 2002). PD-1 is expressed on activated B cells, T cells, and myeloid cells (Agata, et al. 1996; Finger, et al. 1997). The antibody-based therapy and other immunotherapy can target immune checkpoint proteins such as programmed death 1 polypeptide (PD-1) of lymphocytes and block protein-ligand interaction (Bennett, et al., 2003; Blank, et al. 2005; Mahoney et al. 2015). PD-L1 (B7-H1) is the first ligand discover for PD-1 (Dong, et al. 2002, 2003; Konishi, et al., 2004). PD-L2 is a second ligand for PD-1 and inhibits T cell activation (Latchman, et al 2001). An anti-PD-1 antibody can bind to PD-1 and blocks the binding of PD-L1 and/or PD-L2 to PD-1. Therefore, the activation of checkpoint proteins from their ligands is inhibited. Moreover, the treatment of several types of tumors, including melanoma and lung cancers, has been shown to display impressive response upon antibody treatment targeting PD-1 (Carvalho, et al., 2016). Currently, two humanized anti-PD-1 antibodies are approved by the regulatory bodies (Wolchok, et al., 2015, 2013).

The PD-1/PD-L1 signaling pathway which plays a key role in tumor immunity has become a molecular target in tumor immunotherapy. Anti-tumor activity of T cells by tumor cells can be significantly enhanced by blocking the PD-1/PD-L1 signaling pathway. Monoclonal antibodies targeting PD-1 that have been approved by FDA include Keytruda (Pembrolizumab) from Merck and Opdivo (Nivolumab) from Bristol-Myers Squibb. Monoclonal antibodies targeting PD-L1 include Tecentriq (Atezolizumab) from Roche, Bavencio (Avelumab) produced by Pfizer and Merck, and Imfinzi (Durvalumab) from AstraZeneca. These monoclonal antibodies specific for PL-1/PD-L1 have already achieved promising clinical results particularly on melanoma and metastatic squamous non-small cell lung cancer and other high mutation burdens of “hot” tumors.

However, therapeutic antibodies to PD-1/PD-L1 axis do not have widely clinical indications in many ways, such as the tumor response only in a fraction of cases and tumor types. The biggest limitation of the immunotherapy is that the PD-1/PD-L1 monospecific treatment only showed a limited effectiveness in the “cold” tumors like glioblastoma pancreatic, prostate, and ovarian cancers etc. Further advances in the effectiveness of immunotherapy to treat cancer will require targeting antitumor immune response at different mechanisms, which may be accomplished in combination with multiple agents.

CD47 is a 50 kDa multipass transmembrane protein that acts as a ‘marker of self’ and is widely expressed on cell surface (also called integrin-associated protein). It interacts with the myeloid inhibitory immuno receptor SIRP alpha (also termed CD172a or SHPS-1). This interaction of SIRP alpha with CD47 controls the effector functions of innate immune cells such as host cell phagocytosis (Steven E. Kauder et al, 2018). CD47 expression and/or activity is associated with a number of diseases and disorders. Accordingly, there exists a need for therapies that target CD47, as well as better methods for making such therapies.

The CD47-SIRP-alpha interaction sends a “don't eat me” signal to the phagocytic cells. Therefore, blocking the CD47-SIRP-alpha interaction with a monoclonal antibody can provide an effective anti-cancerous treatment, i.e., increasing phagocytosis of CD47-expressing cells by macrophages (reviewed in Chao, et al, 2012 Curr Opin Immunol, 24(2): 225-32), for improved uptake and removal of cancer cells by the host's immune system. This mechanism is effective in leukemias, lymphomas, and many other types of solid tumors. Furthermore, these CD47-blocking antibodies have been shown to synergize with other therapeutic antibodies including Rituxan® and Herceptin® in tumor models. The macrophage checkpoint inhibitor 5F9 (anti-CD47 antibody) combined with rituximab showed good activity in patients with aggressive and indolent lymphoma (Advani R et al, 2018).

There exists a need in the art for antibodies that combine the benefits of anti-PD1/PDL1 and anti-CD47 (or an antigen-binding portion thereof) to for improved clinical efficacy on various diseases including cancers.

### SUMMARY OF INVENTION

In an aspect, the present disclosure provides an isolated bispecific antibody or antigen-binding portion thereof, comprising: (a) a first chain comprising a light chain variable (VL) domain comprising a light chain variable region CDR1 comprising the amino acid sequence of SEQ ID NO:3, a light chain variable region CDR2 comprising the amino acid sequence of SEQ ID NO:4, a light chain variable region CDR3 comprising the amino acid sequence of SEQ ID NO:5; and a heavy chain variable (VH) domain comprising a heavy chain variable region CDR1 comprising the amino acid sequence of SEQ ID NO:7, a heavy chain variable region CDR2 comprising the amino acid sequence of SEQ ID NO:8, and a heavy chain variable region CDR3 comprising the amino acid sequence of SEQ ID NO:9, wherein first chain specifically binds to human PD-1(hPD-1) and blocks the interaction between hPD-1 and PD-L1; (b) a second chain comprising a light chain variable (VL) domain comprising a light chain variable region CDR1 comprising the amino acid sequence of SEQ ID NO:12, a light chain variable region CDR2 comprising the amino acid sequence of SEQ ID NO:13, a light chain variable region CDR3 comprising the amino acid sequence of SEQ ID NO:14; and a heavy chain variable (VH) domain comprising a heavy chain variable region CDR1 comprising the amino acid sequence of SEQ ID NO:16, a heavy chain variable region CDR2 comprising the amino acid sequence of SEQ ID NO:17, and a heavy chain variable region CDR3 comprising the amino acid sequence of SEQ ID NO:18; wherein the second chain specifically binds to human CD47 and inhibits its interaction with SIRP-alpha.

In some embodiments of the bispecific antibody, the first chain and the second chain are coupled in a knob-in-hole format through their respective CH3 domain.

In some embodiments, the VL domain and the VH domain of each of the first chain and the second chain are connected by a flexible linker to form a respective scFv.

In some embodiments, of claim 3, there the linker comprises one or more repeat units of GGGS or GGGGS unit.

In another aspect, the present disclosure provides an isolated bispecific antibody or antigen-binding portion thereof, comprising: (a) a first chain comprising a VL domain comprising the amino acid sequence of SEQ ID NO:2 and a VH domain comprising the amino acid sequence of SEQ ID NO:6; and (b) a second chain comprising a VL domain comprising the amino acid sequence of SEQ ID NO:11 and a VH domain comprising the amino acid sequence of SEQ ID NO:15. In some embodiments, the first chain and the second chain are coupled in a knob-in-hole format through their respective CH3 domain. In some embodiments, the VL domain and the VH domain of each of the first chain and the second chain are connected by a flexible linker to form a respective scFv. In some embodiments, the linker comprises one or more units of GGGS or GGGGS unit.

In another aspect, the present disclosure provides an isolated bispecific antibody or antigen-binding portion thereof, comprising: (a) a first chain comprising the amino acid sequence of SEQ ID NO:1; and (b) a second chain comprising the amino acid sequence of SEQ ID NO:10.

The antibody as described in can be a monoclonal antibody.

In another aspect, the present disclosure provides a pharmaceutical composition comprising the antibody or antigen-binding portion, as described herein, and a pharmaceutically acceptable carrier.

In a further aspect, the present disclosure provides a method of treating cancer in a human, comprising the step of administering to the human the antibody or antigen-binding portion according to any of claims 1-10 in an amount effective to treat said cancer.

In a further aspect, a method of enhancing an immune response in a subject in need thereof, comprising the step of administering to the subject an effective amount of the antibody or antigen-binding portion, wherein said response is indicated by activation of antigen presenting cells.

### BRIEF DESCRIPTION OF THE DRAWINGS

FIG. 1 shows SDS-PAGE and SEC assay results of an anti-PD-1/CD47 bispecific antibody of the present invention.

FIG. 2 shows a flow cytometry analysis plots demonstrating the binding of an anti-PD-1/CD47 bispecific antibody of the present invention to PD1 and CD47 targets.

FIG. 3 shows in vivo results of a tumor mouse model using an anti-PD-1/CD47 bispecific antibody of the present invention as an agent as compared to using certain anti-PD-1 and anti-CD47 reference monospecific antibodies.

### DETAILED DESCRIPTION OF THE INVENTION

To improve the clinical efficacy of current PD-1/PD-L1 or CD47 monospecific therapy, the present disclosure provides isolated bispecific antibodies targeting both PD-1 and CD47. In certain embodiments, the antibodies of the disclosed invention are derived from identified heavy and light chain germline sequences and/or comprise identified structural features such as CDR regions comprising identified amino acid sequences. This disclosure provides isolated antibodies, methods of making such antibodies and antigen-binding portions thereof. This disclosure also relates to methods of using the antibodies, such as using the antibodies of the disclosed invention to stimulate immune responses, alone or in combination with other immunostimulatory antibodies. Accordingly, also provided are methods of using the antibodies of the disclosed invention for example, including but not limited to, treating cancer in a human.

The following embodiments and aspects thereof are described and illustrated in conjunction with systems, compositions and methods which are meant to be exemplary and illustrative, not limiting in scope.

### Definitions

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM and most preferably ≤10 nM.

The term “immune response” as used herein can refer to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from an organism of invading pathogens, cells or tissues infected with pathogens, cancerous cells, or, in cases of autoimmunity or pathological inflammation, normal organismal cells or tissues.

An “antigen-specific T cell response” as used herein can refer to responses by a T cell that result from stimulation of the T cell with the antigen for which the T cell is specific. Non-limiting examples of responses by a T cell upon antigen-specific stimulation include, but are not limited to, proliferation and cytokine production (e.g., IL-2 production).

The term “antibody” as used herein can include whole antibodies, F(ab′)2 fragment, diabody, triabody, tetrabody, bispecific antibody, monomeric antibodies and any antigen binding fragment (i.e., “antigen-binding portion”) or single-chain variable region fragment (scFv), or disulfide-stabilized variable region fragment (dsFv) thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, can refer to one or more fragments of an antibody that retain the ability to specifically bind to an antigen (e.g., a PD-1 protein). It has been shown that the antigen-binding function of an antibody can be performed by fragments of a full-length antibody. Examples of binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3.sup.rd ed. 1993); (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as intact antibodies.

An “isolated antibody”, as used herein, can refer to an antibody that is substantially free of other antibodies having different antigenic specificities (e.g., an isolated bispecific antibody that specifically binds a PD-1 protein and CD47 protein can be substantially free of antibodies that specifically bind antigens other than PD-1 and CD47 proteins).

Bispecific antibodies producing cells, e.g., hybridomas, can be selected, cloned and further screened for desirable characteristics, including robust growth, high antibody production and desirable antibody characteristics, as further discussed below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

The terms “monoclonal antibody” or “monoclonal antibody composition” as used herein can refer to a preparation of antibody molecules of single molecular composition. A monoclonal antibody composition displays a single binding specificity and affinity for a particular epitope.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. Additional framework region modifications can be made within the human framework sequences. In another embodiment, the term “humanized antibody” can refer to antibodies in which CDR sequences derived from the germline of another mammalian species, such as a mouse, have been grafted onto human framework sequences. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” can refer to antibodies in which the variable region sequences can be derived from one species and the constant region sequences can be derived from another species, such as an antibody in which the variable region sequences can be derived from a mouse antibody and the constant region sequences can be derived from a human antibody.

As used herein, an antibody or a portion thereof that “specifically binds human PD-1” can refer to an antibody or a portion thereof that binds to a human PD-1 protein (and possibly a PD-1 protein from one or more non-human species) but does not substantially bind to non-PD-1 proteins. Preferably, the binding to a human PD-1 protein with “high affinity,” namely with a EC 50 of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less.

As used herein, an antibody or a portion thereof that “specifically binds human CD47” can refer to an antibody or a portion thereof that binds to a human CD47 protein (and possibly a CD47 protein from one or more non-human species) and can enhance tumor-targeting immune response by activating tumor-specific innate immune response. Preferably, the binding to a human CD47 protein is with “high affinity,” namely with a Kd of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less.

The term “does not substantially bind” to a protein or cells, as used herein, can mean that it cannot bind or does not bind with a high affinity to the protein or cells, i.e., binds to the protein or cells with an EC 50 of 2×10⁻⁶ M or more, more preferably 1×10⁻⁵ M or more, more preferably 1×10⁻⁴ M or more, more preferably 1×10⁻³ M or more, even more preferably 1×10⁻² M or more.

The term “high affinity” for an IgG antibody can refer to an antibody having an EC 50 of 1×10⁻⁶ M or less, more preferably 1×10⁻⁷ M or less, even more preferably 1×10⁻⁸ M or less, even more preferably 1×10⁻⁹ M or less, even more preferably 1×10⁻¹⁰ M or less for a target antigen. However, “high affinity” binding can vary for other antibody isotypes.

As used herein, the term “inhibit” refers to any decrease in, for example a particular action, function, or interaction. For example, a biological function, such as the function of a protein and/or binding of one protein to another, is inhibited if it is decreased as compared to a reference state, such as a control like a wild-type state or a state in the absence of an applied agent. For example, the binding of a PD-1 protein to one or more of its ligands, such as PD-L1 and/or PD-L2, and/or resulting PD-1 signaling and immune effects is inhibited or deficient if the binding, signaling, and other immune effects are decreased due to contact with an agent, such as an anti-PD-1 antibody, in comparison to when the PD-1 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked. For another example, the binding of a CD47 protein to one or more of its ligands, such as, and/or resulting CD47 signaling and immune effects is decreased, if the binding, signaling, and other immune effects are decreased due to contact with an agent, such as an anti-CD47 antibody, in comparison to when the CD47 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked.

The term “subject” can refer to any human or non-human animal. The term “nonhuman animal” includes all vertebrates, e.g., mammals and non-mammals, such as nonhuman primates, sheep, dogs, cats, cows, horses, chickens, rabbits, mice, rats, amphibians, and reptiles, although mammals are preferred, such as non-human primates, sheep, dogs, cats, cows and horses.

The binding of an antibody of the disclosed invention to PD-1/CD47 can be assessed using one or more techniques well established in the art. For example, in a preferred embodiment, an antibody can be tested by ELISA assays.

Preferably, an antibody of the disclosed invention binds to a PD-1 protein with an EC 50 of 5×10⁻⁸ M or less, binds to a PD-1 protein with a EC 50 of 2×10⁻⁸ M or less, binds to a PD-1 protein with a EC 50 of 5×10⁻⁹ M or less, binds to a PD-1 protein with a EC 50 of 4×10⁻⁹ M or less, binds to a PD-1 protein with a EC 50 of 3×10⁻⁹ M or less, binds to a PD-1 protein with a EC 50 of 2×10⁻⁹ M or less, binds to a PD-1 protein with a EC 50 of 1×10⁻⁹ M or less.

Preferably, an antibody of the disclosed invention binds to a CD47 protein with an EC 50 of 5×10⁻⁸ M or less, binds to a CD47 protein with a EC 50 of 2×10⁻⁸ M or less, binds to a CD47 protein with a EC 50 of 5×10⁻⁹ M or less, binds to a CD47 protein with a EC 50 of 4×10⁻⁹ M or less, binds to a CD47 protein with a EC 50 of 3×10⁻⁹ M or less, binds to a CD47 protein with a EC 50 of 2×10⁻⁹ M or less, binds to a CD47 protein with a EC 50 of 1×10⁻⁹ M or less.

B cells or hybridoma expressing the antibodies against the antigen can be isolated and the nucleic acid sequences encoding the heavy chain variable domain (VH) and light chain variable domain (VL) can be obtained by next generation sequencing. The encoded amino acid sequences of VH and VL can be obtained from the nucleic acid sequences. These techniques are well known by the person of ordinary skill in the art.

The CDR regions can be delineated using the Kabat system (Kabat et al. (1991) Sequences of Proteins of Immunological Interest, Fifth Edition, U.S. Department of Health and Human Services, NIH Publication No. 91-3242).

An antibody of the disclosed invention further can be prepared using an antibody having one or more of the VH and/or VL sequences disclosed herein as starting material to engineer a modified antibody, which modified antibody can have altered properties from the starting antibody. An antibody can be engineered by modifying one or more residues within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs can be more diverse between individual antibodies than sequences outside of CDRs.

Because CDR sequences can be responsible for most antibody-antigen interactions, it can be possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al. (1998) Nature 332:323-327; Jones et al. (1986) Nature 321: 522-525; Queen et al. (1989) Proc. Natl. Acad. See. U.S.A. 86: 10029-10033; U.S. Pat. Nos. 5,225,539; 5,530,101; 5,585,089; 5,693,762 and 6,180,370.)

Accordingly, in one aspect of the present disclosure, an isolated bispecific antibody or antigen-binding portion thereof is provided, which comprises: (a) a first chain comprising a light chain variable (VL) domain comprising a light chain variable region CDR1 comprising the amino acid sequence of SEQ ID NO:3, a light chain variable region CDR2 comprising the amino acid sequence of SEQ ID NO:4, a light chain variable region CDR3 comprising the amino acid sequence of SEQ ID NO:5; and a heavy chain variable (VH) domain comprising a heavy chain variable region CDR1 comprising the amino acid sequence of SEQ ID NO:7, a heavy chain variable region CDR2 comprising the amino acid sequence of SEQ ID NO:8, and a heavy chain variable region CDR3 comprising the amino acid sequence of SEQ ID NO:9, wherein first chain or the antigen-binding portion thereof specifically binds to human PD-1(hPD-1) and blocks the interaction between hPD-1 and PD-L1; (b) a second chain comprising a light chain variable (VL) domain comprising a light chain variable region CDR1 comprising the amino acid sequence of SEQ ID NO:12, a light chain variable region CDR2 comprising the amino acid sequence of SEQ ID NO:13, a light chain variable region CDR3 comprising the amino acid sequence of SEQ ID NO:14; and a heavy chain variable (VH) domain comprising a heavy chain variable region CDR1 comprising the amino acid sequence of SEQ ID NO:16, a heavy chain variable region CDR2 comprising the amino acid sequence of SEQ ID NO:17, and a heavy chain variable region CDR3 comprising the amino acid sequence of SEQ ID NO:18; wherein the second chain or the antigen-binding portion thereof specifically binds to human CD47 and inhibits its interaction with SIRP-alpha.

In some embodiments, the first chain and the second chain of the bispecific antibody are coupled in a knob-in-hole format through their respective CH3 domain.

In some embodiments, the VL domain and the VH domain of each of the first chain and the second chain are connected by a flexible linker to form a respective scFv. For example, the linker can include (G)nS, such as one or more repeat units of GGGS or GGGGS unit.

In another aspect, the present disclosure provides an isolated bispecific antibody or antigen-binding portion thereof, which includes: (a) a first chain comprising a VL domain comprising the amino acid sequence of SEQ ID NO:2 and a VH domain comprising the amino acid sequence of SEQ ID NO:6; and (b) a second chain comprising a VL domain comprising the amino acid sequence of SEQ ID NO:11 and a VH domain comprising the amino acid sequence of SEQ ID NO:15.

In some of these embodiments, the first chain and the second chain are coupled in a knob-in-hole format through their respective CH3 domain.

In some of these embodiments, the VL domain and the VH domain of each of the first chain and the second chain are connected by a flexible linker to form a respective scFv. For example, the linker can include (G)nS, such as one or more repeat units of GGGS or GGGGS unit.

In another aspect, the present disclosure provides an isolated bispecific antibody or antigen-binding portion thereof, comprising: (a) a first chain comprising the amino acid sequence of SEQ ID NO:1; and (b) a second chain comprising the amino acid sequence of SEQ ID NO:10.

The isolated bispecific antibody or antigen-biding portion of described can be a monoclonal antibody.

Antibody protein sequences are compared against a compiled protein sequence database using one of the sequence similarity searching methods called the Gapped BLAST (Altschul et al. (1997), supra), which is well known to those skilled in the art. The compositions and methods of the presently disclosed invention are not limited to variants of the exemplary sequences disclosed herein but include those having at least 90%, at least 95% and at least 99% sequence identity to an exemplary sequence disclosed herein.

A nucleic acid molecule encoding the heavy or entire light chain of a bispecific antibody or portions thereof can be isolated from any source that produces such an antibody. Methods of isolating mRNA encoding an antibody are well-known in the art. See, e.g., Sambrook et al. The mRNA may be used to produce cDNA for use in the polymerase chain reaction (PCR) or cDNA cloning of antibody genes. In a preferred embodiment, the nucleic acid molecule is isolated from a hybridoma that has as one of its fusion partners a human immunoglobulin producing cell from a non-human transgenic animal. In another embodiment, the nucleic acid can be isolated from a non-human, non-transgenic animal. The nucleic acid molecules isolated from a non-human, nontransgenic animal may be used, e.g., for humanized antibodies.

In another aspect, the present disclosure provides a pharmaceutical composition comprising one or more antibodies of the present invention formulated together with a pharmaceutically acceptable carrier. The composition may optionally contain one or more additional pharmaceutically active ingredients, such as another antibody or a drug. The pharmaceutical compositions of the disclosed invention also can be administered in combination therapy with, for example, another immunostimulatory agent, an anti-cancer agent, an antiviral agent, or a vaccine, such that the bispecific antibody enhances the immune response stimulated by the vaccine.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the bispecific antagonist antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results.

In certain embodiments, the antibody compositions active compound may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

In certain embodiments, a bispecific antibody of the disclosed invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the bispecific antibodies can be incorporated with excipients and used in the form of ingestible tablets, buccal tablets, troches, lozenge, capsules, elixirs, suspensions, syrups, wafers, and the like. To administer a compound of the disclosed invention by other than parenteral administration, it may be necessary to coat the compound with, or co-administer the compound with, a material to prevent its inactivation.

Additional active compounds also can be incorporated into the disclosed compositions. In certain embodiments, a bispecific antibody of the disclosed invention is co-formulated with and/or co-administered with one or more additional therapeutic agents. These agents include, without limitation, antibodies that bind other targets (e.g., antibodies that bind one or more growth factors or cytokines or their cell surface receptors, such as anti-CTL4-antibody), antineoplastic agents, antitumor agents, chemotherapeutic agents, peptide analogues that activate CD40, soluble CD40L, one or more chemical agents that activates CD40, CpG oligodeoxynucleotides and/or other agents known in the art that can enhance an immune response against tumor cells, e.g., IFN-1, IL-2, IL-8, IL-12, IL-15, IL-18, IL-23, IFN-y, and GM-CSF. Such combination therapies may require lower dosages of the antibody as well as the co-administered agents, thus avoiding possible toxicities or complications associated with the various monotherapies.

Antagonist the bispecific antibodies of the disclosed invention and compositions comprising them also may be administered in combination with other therapeutic regimens, in particular in combination with radiation treatment.

The pharmaceutical compositions of the disclosed invention can include pharmaceutically acceptable salts. A “pharmaceutically acceptable salt” can refer to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration can be required.

An exemplary, non-limiting range for a therapeutically or prophylactically effective amount of an antibody or antibody portion of the disclosed invention is 0.025 to 50 mg/kg, more preferably 0.1 to 50 mg/kg, more preferably 0.1 to 25 mg/kg, 0.1 to 10 mg/kg and 0.1 to 3 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Bispecific antibodies or antigen-binding fragments can be produced by a variety of methods including fusion of hybridomas or linking of Fab′ fragments. See, e.g., Songsivilai & Lachmarm, Clin. Exp. Immunol. 79: 315-321 (1990), Kos-telny et al., J. Immunol. 148:1547-1553 (1992). In addition, bispecific antibodies can be formed as “diabodies” or “Janusins.”

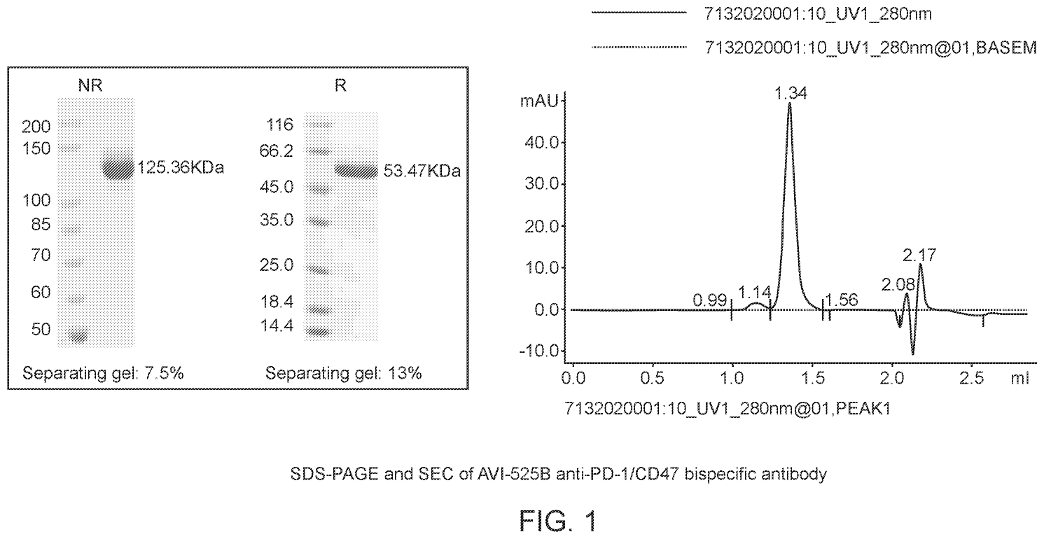

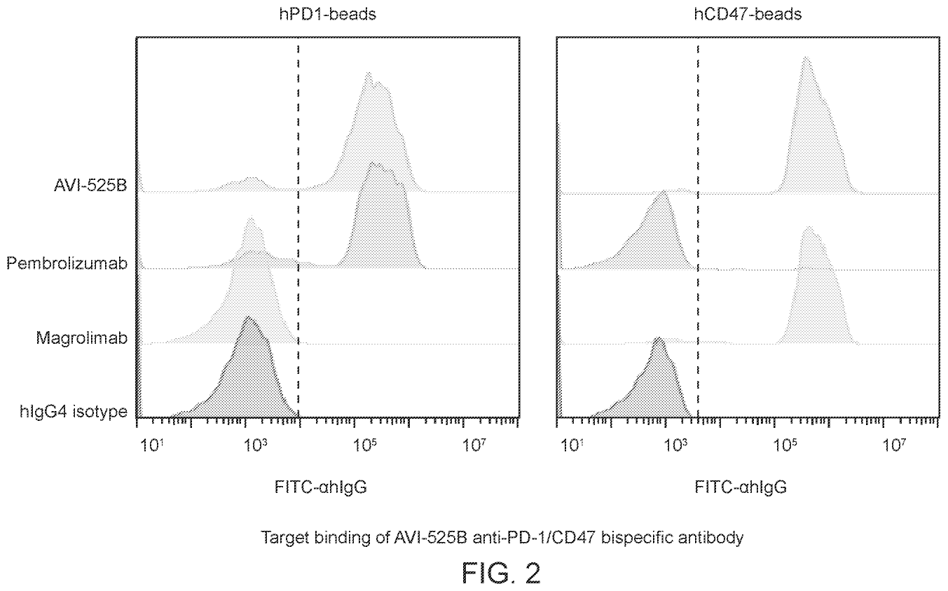

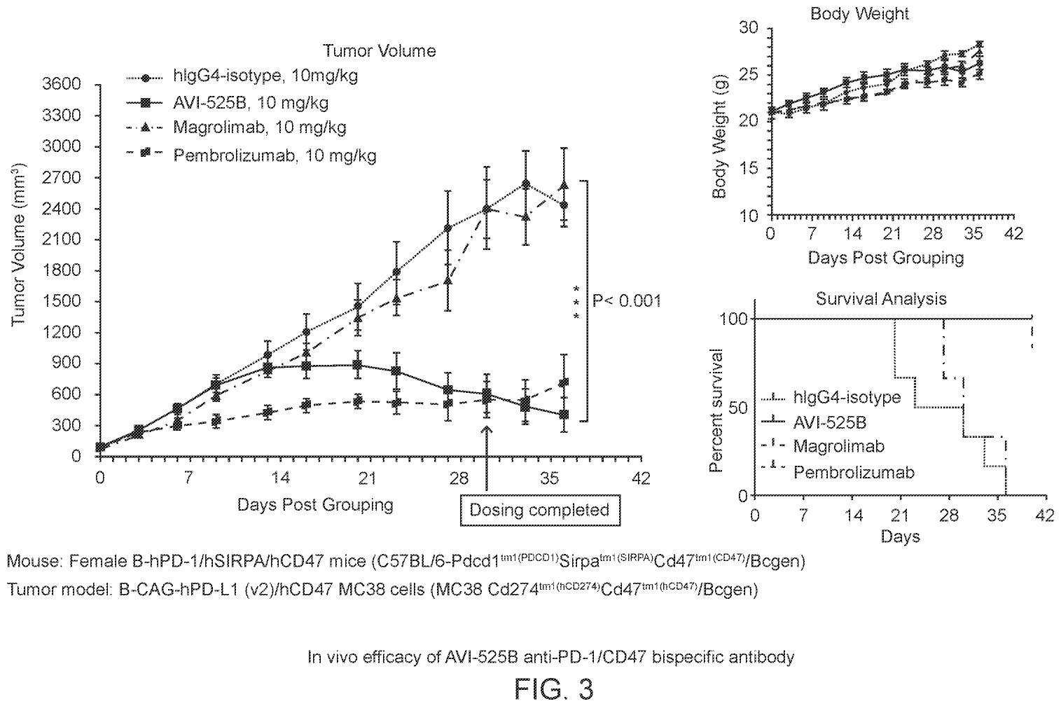

Example 1: A bispecific anti-PD-1/CD47 antibody was designed and generated (Clone #: AVI-525B, the sequence of its components are provided in the accompanying Sequence Listing). The VH and VL against PD-1 and CD47 were separately developed, and the scFv for either anti-PD-1 or anti-CD47 chain includes the respective VL linked with a linker of (GGGGS) 4 to the corresponding VH. The anti-PD-1 scFv was fused with a hinge, CH2, and CH3-knob of the bispecific antibody. The anti-CD47 scFv was fused with a hinge, CH2, and CH3-hole of bispecific antibody. The bispecific antibody was produced with either HEK239 or CHO cells. The developability of bispecific antibody was accessed by size exclusion chromatography (SEC) and SDS-PAGE (FIG. 1). The binding ability of bispecific antibody to PD-1 and CD47 proteins was confirmed by flow cytometry (FIG. 2). The in vivo efficacy study was conducted in the female B-hPD-1/hSIRPA/hCD47 mice (C57BL/6-^{Pdcd1tml(PDCD1)}Sirpa^{tml(SIRPA)}Cd47^{tml(cD47)/}Bcgen) with tumor model of B-CAG-hPD-L1 (v2)/hCD47 MC38 cells (MC38 Cd274^{tml(hCD274)Cd47tml(hCD47)/}Bcgen). The treatment started at tumor size around 85 mm³. Mice were treated three times per week with isotype, reference antibody (Magrolimab and Pembrolizumab), and anti-PD-1/CD47 bispecific antibody. The dose of 10 mg/kg was administrated by intraperitoneal injection. The treatment period was 5 weeks. The tumor size and body weight were measure twice per week. The results (FIG. 3) demonstrated a superior efficacy of the bispecific antibody compared with the monospecific treatment with the clinical reference antibodies, Pembrolizumab (anti-PD-1) and Magrolimab (anti-CD47).

Although the above invention has been described in some detail by way of illustration and example for purposes of clarity of understanding, it is apparent to those skilled in the art that certain changes and modifications will be practiced. Therefore, the description and examples should not be construed as limiting the scope of the disclosed invention.

The disclosures of all publications, patents, patent applications and published patent applications referred to herein by an identifying citation are hereby incorporated herein by reference in their entirety. All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

What is claimed is:

1. An isolated bispecific antibody or antigen-binding portion thereof, comprising:
(a) a first chain comprising a light chain variable (VL) domain comprising a light chain variable region CDR1 comprising the amino acid sequence of SEQ ID NO:3, a light chain variable region CDR2 comprising the amino acid sequence of SEQ ID NO:4, a light chain variable region CDR3 comprising the amino acid sequence of SEQ ID NO:5; and a heavy chain variable (VH) domain comprising a heavy chain variable region CDR1 comprising the amino acid sequence of SEQ ID NO:7, a heavy chain variable region CDR2 comprising the amino acid sequence of SEQ ID NO:8, and a heavy chain variable region CDR3 comprising the amino acid sequence of SEQ ID NO:9, wherein first chain specifically binds to human PD-1(hPD-1) and blocks the interaction between hPD-1 and PD-L1;
(b) a second chain comprising a light chain variable (VL) domain comprising a light chain variable region CDR1 comprising the amino acid sequence of SEQ ID NO:12, a light chain variable region CDR2 comprising the amino acid sequence of SEQ ID NO:13, a light chain variable region CDR3 comprising the amino acid sequence of SEQ ID NO:14; and a heavy chain variable (VH) domain comprising a heavy chain variable region CDR1 comprising the amino acid sequence of SEQ ID NO:16, a heavy chain variable region CDR2 comprising the amino acid sequence of SEQ ID NO:17, and a heavy chain variable region CDR3 comprising the amino acid sequence of SEQ ID NO:18; wherein the second chain specifically binds to human CD47 and inhibits its interaction with SIRP-alpha.

2. The isolated bispecific antibody or antigen-biding portion thereof, of claim 1, wherein the first chain and the second chain are coupled in a knob-in-hole format through their respective CH3 domain.

3. The isolated bispecific antibody or antigen-biding portion thereof, of claim 1, wherein the VL domain and the VH domain of each of the first chain and the second chain are connected by a flexible linker to form a respective scFv.

4. The isolated bispecific antibody or antigen-biding portion thereof, of claim 3, wherein the linker comprises one or more repeat units of GGGS or GGGGS unit.

5. An isolated bispecific antibody or antigen-binding portion thereof, comprising:
(a) a first chain comprising a VL domain comprising the amino acid sequence of SEQ ID NO:2 and a VH domain comprising the amino acid sequence of SEQ ID NO:6; and
(b) a second chain comprising a VL domain comprising the amino acid sequence of SEQ ID NO:11 and a VH domain comprising the amino acid sequence of SEQ ID NO:15.

6. The isolated bispecific antibody or antigen-biding portion thereof, of claim 5, wherein the first chain and the second chain are coupled in a knob-in-hole format through their respective CH3 domain.

7. The isolated bispecific antibody or antigen-biding portion thereof, of claim 5, wherein the VL domain and the VH domain of each of the first chain and the second chain are connected by a flexible linker to form a respective scFv.

8. The isolated bispecific antibody or antigen-biding portion thereof, of claim 7, wherein the linker comprises one or more units of GGGS or GGGGS unit.

9. An isolated bispecific antibody or antigen-binding portion thereof, comprising:
(a) a first chain comprising the amino acid sequence of SEQ ID NO:1; and
(b) a second chain comprising the amino acid sequence of SEQ ID NO:10.

10. The isolated bispecific antibody or antigen-biding portion of any of the foregoing claims, wherein the antibody is a monoclonal antibody.

11. A pharmaceutical composition comprising the antibody or antigen-binding portion according to any of claims 1-10, and a pharmaceutically acceptable carrier.

12. A method of treating cancer in a human, comprising the step of administering to the human the antibody or antigen-binding portion according to any of claims 1-10 in an amount effective to treat said cancer.

13. A method of enhancing an immune response in a subject in need thereof, comprising the step of administering to the subject an effective amount of the antibody or antigen-binding portion according to any of claims 1-10, wherein said response is indicated by activation of antigen presenting cells.


SEQUENCE LISTING (18 sequences)

SEQ ID NO: 1 - protein, 480 aa, Artificial Sequence
  Synthetic
DIVMTQSPSSLSTSVGDRVTITCKASQSVSNDVTWYQQKPGKSPKLLIYYAAKRYTGVPSRFSGSGYGTDFTFTISSVQPEDIAVYFCQQNYNSPYTFGGGTKLEIKGGGGSGGGGSGGGGSGGGGSDIQLVESGGDLVKPGGSLKLSCAASGFTFSSYGMSWVRQTPDKRLEWVASISNGGNTYCSDSVKGRFTISRDNARNILYLQMSSLKSEDTAMYYCARLYGSNYLYWYIDVWGQGTLVTVSSEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 107 aa, Artificial Sequence
  Synthetic
DIVMTQSPSSLSTSVGDRVTITCKASQSVSNDVTWYQQKPGKSPKLLIYYAAKRYTGVPSRFSGSGYGTDFTFTISSVQPEDIAVYFCQQNYNSPYTFGGGTKLEIK

SEQ ID NO: 3 - protein, 6 aa, Artificial Sequence
  Synthetic
QSVSND

SEQ ID NO: 4 - protein, 3 aa, Artificial Sequence
  Synthetic
YAA

SEQ ID NO: 5 - protein, 9 aa, Artificial Sequence
  Synthetic
QQNYNSPYT

SEQ ID NO: 6 - protein, 119 aa, Artificial Sequence
  Synthetic
QLVESGGDLVKPGGSLKLSCAASGFTFSSYGMSWVRQTPDKRLEWVASISNGGNTYCSDSVKGRFTISRDNARNILYLQMSSLKSEDTAMYYCARLYGSNYLYWYIDVWGQGTLVTVSS

SEQ ID NO: 7 - protein, 8 aa, Artificial Sequence
  Synthetic
GFTFSSYG

SEQ ID NO: 8 - protein, 7 aa, Artificial Sequence
  Synthetic
ISNGGNT

SEQ ID NO: 9 - protein, 15 aa, Artificial Sequence
  Synthetic
ARLYGSNYLYWYIDV

SEQ ID NO: 10 - protein, 475 aa, Artificial Sequence
  Synthetic
DIKMTQSPSSMSASVGDRVTITCQASQDINSYLSWFQQKPGKSPKTLIYRVNRLVTGVPSRFSGSGSGQDYSLTISSLQPEDIGIYYCLQYDEFPLTFGGGTKLELKGGGGSGGGGSGGGGSGGGGSEVKLVESGGGLVKPGGSLKLSCAASGFAFSTYDMSWIRQTPEKRLEWVATISTGGTYTYYPDSVKGRFTISRDNAKNTLYLQMSSLKSEDTALYYCSRRPYYFDYWGQGTTLTVSSEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 107 aa, Artificial Sequence
  Synthetic
DIKMTQSPSSMSASVGDRVTITCQASQDINSYLSWFQQKPGKSPKTLIYRVNRLVTGVPSRFSGSGSGQDYSLTISSLQPEDIGIYYCLQYDEFPLTFGGGTKLELK

SEQ ID NO: 12 - protein, 6 aa, Artificial Sequence
  Synthetic
QDINSY

SEQ ID NO: 13 - protein, 3 aa, Artificial Sequence
  Synthetic
RVN

SEQ ID NO: 14 - protein, 9 aa, Artificial Sequence
  Synthetic
LQYDEFPLT

SEQ ID NO: 15 - protein, 114 aa, Artificial Sequence
  Synthetic
KLVESGGGLVKPGGSLKLSCAASGFAFSTYDMSWIRQTPEKRLEWVATISTGGTYTYYPDSVKGRFTISRDNAKNTLYLQMSSLKSEDTALYYCSRRPYYFDYWGQGTTLTVSS

SEQ ID NO: 16 - protein, 7 aa, Artificial Sequence
  Synthetic
GFAFSTY

SEQ ID NO: 17 - protein, 8 aa, Artificial Sequence
  Synthetic
ISTGGTYT

SEQ ID NO: 18 - protein, 9 aa, Artificial Sequence
  Synthetic
SRRPYYFDY